(19) United States
(12) Patent Application Publication
(10) Pub. No.: US 2022/0226527 A1
(43) Pub. Date: Jul. 21, 2022

(54) SPRAY BOTTLE INSERT INCLUDING CHLORINE DIOXIDE MICRO REACTOR UTILIZING MEMBRANE PACKAGING

(71) Applicant: Selective Micro Technologies, LLC, Dublin, OH (US)

(72) Inventor: Jeff Thomas, Plain City, OH (US)

(21) Appl. No.: 17/577,976

(22) Filed: Jan. 18, 2022

Related U.S. Application Data
(60) Provisional application No. 63/137,933, filed on Jan. 15, 2021.

(51) Int. Cl. A61L 2/26; A61L 2/18; A61L 2/28

(52) U.S. Cl. CPC A61L 2/26; A61L 2101/06; A61L 2/28; A61L 2/18

(57) ABSTRACT

Apparatuses for generation of a sterilizing agent, for example chlorine dioxide, in a spray bottle, methods of forming an apparatus, and methods of use thereof are provided. The apparatus may include a tube housing a packet containing a reactant disposed within the interior of the packet. The tube prevents overexpansion of the packet in the presence of an initiating agent. The apparatus may include an openable capsule that houses a packet containing a reactant disposed within the interior of the packet. The capsule includes holes for the transmission of liquid or humid air.

This application claims priority under 35 U.S.C. § 119 to U.S. Provisional Application No. 63/137,933, which was filed on Jan. 15, 2021. The entire contents of the aforementioned application are incorporated herein by reference in its entirety.

The present disclosure relates to an apparatus for generating a cleaning solution within a spray bottle. In some embodiments, the cleaning solution includes chlorine dioxide. More particularly, the present disclosure is directed to an apparatus housing at least one packet containing a reactant that generates a sterilizing agent in the presence of an initiating agent.

Sterilizing gases such as chlorine dioxide are useful disinfectants in many industries. For example, chlorine dioxide can be used to wash meat and food products to remove food borne pathogens and to prolong shelf life. Chlorine dioxide can also be used to sterilize high traffic areas such as hotels, hospitals, livery vehicles, veterinary clinics, restaurants, office buildings, municipal buildings, schools, and the like.

Chlorine dioxide is often dispersed in a medium such as water. The resulting chlorine dioxide and water mixture may be released out of a container such as a spray bottle. Spray bottles are widely used in both households and commercial settings.

Use of chlorine dioxide can prompt health concerns. For example, chlorine dioxide that is improperly generated or stored can be explosive or ignite. Therefore, many current solutions generate chlorine dioxide within the spray bottle before desired use, for example by adding water to a packet containing a reactant. However, the packets swell when exposed to water and cannot be removed through the neck of the spray bottle. The spray bottle must then be discarded, which is wasteful and economically inefficient.

Therefore, there exists an unmet need for the safe, effective, and repeatable generation of sterilizing agents such as chlorine dioxide within a conventional spray bottle. Accordingly, the present disclosure provides for an apparatus for generating sterilizing agents such as chlorine dioxide, methods of forming the apparatus, and methods of use thereof.

The present disclosure provides various embodiments of an apparatus including a hollow cylinder having a closed end and an open end. The cylinder includes a plurality of holes disposed on a body of the cylinder. The apparatus further includes at least one packet disposed within the cylinder, and at least one packet contains one or more reactants.

In some embodiments, the apparatus further includes a packet containing a desiccant. The packet is disposed within the cylinder. In some embodiments, the plurality of holes allows entry of a liquid into the cylinder. In further embodiments, the cylinder is sufficiently rigid to prevent expansion of the packet beyond the body of the cylinder after entry of the liquid.

In some embodiments, the cylinder is configured to be insertable through a neck of a spray bottle. In further embodiments, the cylinder is mounted to a straw of the spray bottle. In further embodiments, the cylinder is mounted to the straw with at least one clip.

In some embodiments, the apparatus further includes a pH indicator attached to the cylinder. In further embodiments, the pH indicator is a cap removably attached to the open end of the cylinder. In some embodiments, the pH indicator is a tether attached to a cap removably attached to the open end of the cylinder. In some embodiments, the apparatus further includes a cap removably attached to the open end of the cylinder.

The present disclosure provides various embodiments of a method of preparing a disinfecting agent. The method includes selecting a hollow cylinder having a closed end and an open end; inserting and securing a packet within the cylinder; and introducing a liquid into the cylinder. The cylinder includes a plurality of holes disposed on a body of the cylinder. The packet contains a reactant.

In some embodiments, the method further includes inserting the cylinder into a spray bottle. In further embodiments, the cylinder is inserted through a neck of the spray bottle. In some embodiments, the method further includes securing the cylinder within the spray bottle. In further embodiments, the cylinder is secured with at least one clip. In some embodiments, the cylinder is secured to a straw of the spray bottle. In further embodiments, the disinfecting agent is released through the straw out of the spray bottle.

In some embodiments, the liquid is introduced through at least some of the plurality of holes. In some embodiments, the cylinder is sufficiently rigid to prevent expansion the packet beyond the body of the cylinder after introducing of the liquid. In some embodiments, the liquid is water.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1A is a perspective view of a tube housing a packet for generating a sterilizing agent, according to some embodiments.

FIG. 1B is a perspective view of a plastic-wrapped tube housing a packet for generating a sterilizing agent, according to some embodiments.

FIG. 2 is a side view of a tube housing a packet for generating a gas, according to some embodiments.

FIG. 3 is a side view depicting a cross section cut of a tube housing a packet for generating a sterilizing agent depicted in FIG. 8, according to some embodiments.

FIG. 4 is a top view of a tube housing a packet for generating a sterilizing agent, according to some embodiments.

FIG. 5 is a bottom view of a tube housing a packet for generating a sterilizing agent, according to some embodiments.

FIG. 6 is an exploded view of a tube housing a packet for generating a sterilizing agent, according to some embodiments.

FIG. 7 is an exploded view depicting the process of loading a tube housing a packet for generating a sterilizing agent, according to some embodiments.

FIG. 8 is an internal view of a packet situated within a tube, according to some embodiments.

FIG. 9 graphically depicts use of a tube housing a packet for generating a sterilizing agent, according to some embodiments.

FIG. 10 is a mock-up microscopic view of an initiating agent entering a packet containing a reactant, according to some embodiments.

FIG. 11 depicts the section cut line of a spray bottle for the views depicted in FIGS. 12A-12C.

FIG. 12A depicts a first embodiment of a tube housing a packet for generating a sterilizing agent situated in a spray bottle.

FIG. 12B depicts a second embodiment of a tube housing a packet for generating a sterilizing agent situated in a spray bottle.

FIG. 12C depicts a third embodiment of a tube housing a packet for generating a sterilizing agent situated in a spray bottle.

FIG. 13 is a close-up side view of the embodiment of FIG. 12B.

FIGS. 14A, 14B, and 14C depict a pH indicator cap as part of a tube housing a packet for generating a sterilizing agent, according to some embodiments.

FIG. 15 depicts a pH indicator tethered to a tube housing a packet for generating a sterilizing agent, according to some embodiments.

FIG. 16 depicts a pH indicator tethered to a tube housing a packet for generating a sterilizing agent situated within a spray bottle, according to some embodiments.

FIG. 17 depicts labeled dimensions of a conventional spray bottle.

FIG. 18 is a perspective view of a capsule for housing a packet for generating a sterilizing agent, according to some embodiments.

FIG. 19 is a front view of a capsule for housing a packet for generating a sterilizing agent, according to some embodiments.

FIG. 20 is a rear view of a capsule for housing a packet for generating a sterilizing agent, according to some embodiments.

FIG. 21 is a right side view of a capsule for housing a packet for generating a sterilizing agent, according to some embodiments.

FIG. 22 is a left side view of a capsule for housing a packet for generating a sterilizing agent, according to some embodiments.

FIG. 23 is a bottom view of a capsule for housing a packet for generating a sterilizing agent, according to some embodiments.

FIG. 24 is a top view of a capsule for housing a packet for generating a sterilizing agent, according to some embodiments.

FIG. 25 is a section view of the capsule of FIG. 18.

FIG. 26 is a perspective view of a packet being loaded into an open capsule, according to some embodiments.

FIG. 27 is an enlarged view of a detent of a rail for locking the capsule, according to some embodiments.

FIG. 28 is an enlarged view of a notch of a channel for locking the capsule, according to some embodiments.

### DETAILED DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Similarly, the use of the term “comprising,” as well as other forms, such as “comprises,” is also not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

A “conventional spray bottle” is defined as a 16 or 32 ounce bottle having a straw that draws a product within the body of the bottle through a nozzle. Such conventional spray bottles are widely available for sale for household and commercial use.

The disclosed apparatus provides an easily replaceable mechanism for generating a gas, for example chlorine dioxide. The apparatus includes a tube containing a durable packet that safely houses a reactant and is configured to permit the introduction of an initiating agent, such as water, from outside the packet. The tube has sufficient strength to contain the packet within the boundaries of the tube as the packet expands after the introduction of the initiating agent.

Immediate mixing of the reactant within the packet with the initiating agent produces a sterilizing agent that provides hospital-grade disinfection (99.9999% reduction in pathogens). By comparison, typical household cleaners operate as a sanitizer (99.9% reduction in pathogens). However, over time the sterilizing agent produced in the disclosed apparatus loses effectiveness and operates as a sanitizer rather than a hospital-grade disinfectant. Therefore, an apparatus designed to facilitate mixing of the reactant in the packet with water immediately before use is useful.

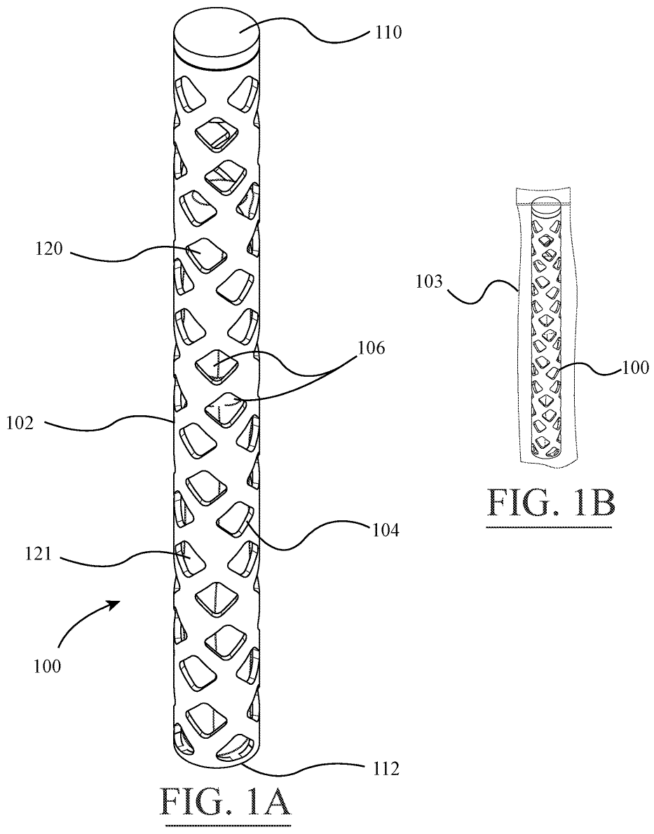

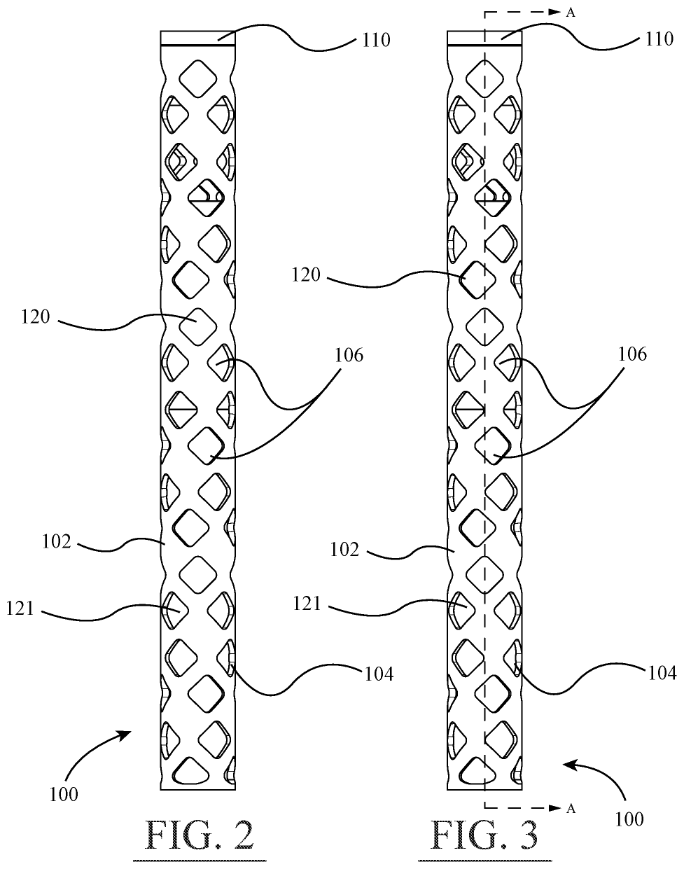

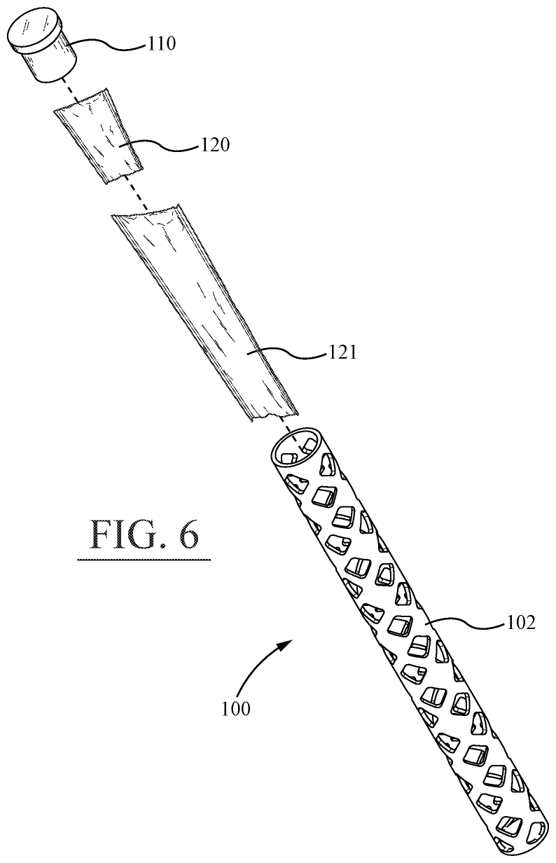

FIG. 1A is a perspective view of, FIG. 2 is a side view of, and FIG. 6 is an exploded view of a tube housing a packet for generating a sterilizing agent, according to some embodiments. The tube 102 is a possible embodiment of an apparatus 100 configured to house at least one desiccant packet 120 and reactant packet 121. The desiccant packet 120 stabilizes the apparatus 100 for a longer shelf life. The desiccant within packet 120 may be any conventional desiccant know to those of ordinary skill in the art.

One of ordinary skill in the art would appreciate that the wall 104 of apparatus 100 may possess a circular, rectangular, triangular, or other cross-sectional shape. For example, the tube 102 may be a hollow cylinder. The wall 104 includes a plurality of holes 106 configured to allow the entrance and exit of fluids. Upon entry of an initiating agent fluid, such as water, into the reactant packet 121, the ensuing reaction between the fluid and reactant may cause the reactant packet 121 to expand in size. In the absence of another structure, reactant packet 121 would expand to a size larger than the neck of a conventional spray bottle. At such an expanded size, removal of a used reactant packet 121 would be impossible without destroying the spray bottle, necessitating the purchase of a new bottle.

Tube 102 may be composed of a compound with sufficient strength to restrict expansion of the reactant packet 121. And tube 102 may be sized to fit within a neck of a spray bottle. Therefore, a used reactant packet 121, stored within tube 102, can be evacuated from the spray bottle after use. The spray bottle may then be reused and another apparatus 100 may be inserted into the bottle. In some embodiments, the apparatus 100 is composed of recyclable material and may be recycled after use.

The packet 121 may be composed of a hydrophobic material. For example, the packet 121 may be composed of polytetrafluoroethylene (PTFE). PTFE offers advantages compared to other possible packet 121 materials. Specifically, PTFE is more robust due to its high hydrophobicity and does not become embrittled during gas generation. In some embodiments, the packet 121 is composed of a single layer of hydrophobic material.

In some embodiments, the hydrophobic material is porous. In further embodiments, the pores may be sized between 0.01 micrometers and 3.00 micrometers, between 0.03 micrometers and 2.00 micrometers, between 0.05 micrometers and 1.00 micrometers, or any range in between. The pores are of suitable size to allow passage of an initiating agent and a generated sterilizing agent through the packet 121 material.

In some embodiments, the reactant is in a solid form. Selection of the reactant determines the sterilizing agent that will be generated once the apparatus 100 is exposed to water. For example, a reactant composed of a combination of sodium chloride and citric acid would generate chlorine dioxide in the presence of water. Reactants may also be chosen to generate chlorine dioxide, carbon dioxide, oxygen, nitrogen, argon, helium, calcium carbonate, or a combination thereof. In some embodiments, a combination of carbon dioxide and chlorine dioxide is generated.

In some embodiments, the reactant fills a portion of the interior of the packet 121. For example, the reactant may fill between 10-90%, 20-60%, 30-50%, or any percentage in between, of the interior of the packet 121. The remaining volume of the interior of the packet 121 that is not filled with reactant may contain air.

The apparatus 100 may include a removable cap 110 situated at one end of the apparatus 100. The apparatus 100 may include a sealed end situated at the opposing end of the apparatus 100. In some embodiments, the sealed end 112 provides a base for a straw of a spray bottle situated within the tube 102.

In some embodiments, the tube 102 may be shrink-wrapped for sale. FIG. 1B is a perspective view of plastic-wrapped tube housing a pouch for generating a sterilizing agent, according to some embodiments. The plastic wrap 103 may be sealed and fully encompasses the tube 102. In alternate embodiments, the apparatus 100 is loaded within a spray bottle and is not wrapped in plastic. The bottle may be sealed at a threaded portion of the bottle to maintain a sterile environment for the apparatus 100.

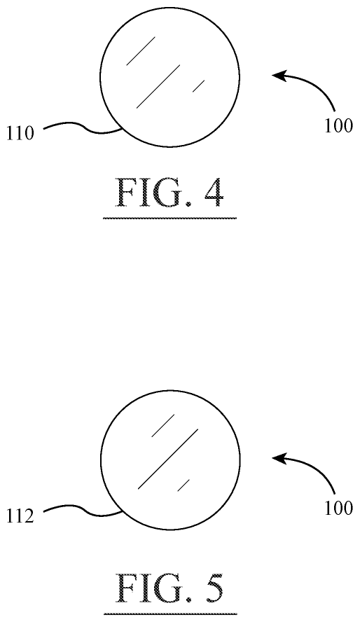

FIG. 4 is a top view of a tube housing a packet for generating a sterilizing agent, according to some embodiments. FIG. 4 illustrates a top view of cap 110 specifically. Cap 110 may be used to retain packets 120 and 121 within the tube 102 while the apparatus 100 is stored before use or sale. Before use, cap 100 can be removed to allow entry of a straw of a spray bottle into the tube 102.

FIG. 5 is a bottom view of a tube housing a packet for generating a gas, according to some embodiments. FIG. 5 illustrates a bottom view of sealed end 112. The sealed end 112 functions to prevent the packets 120, 121 from passing through the bottom of tube 102.

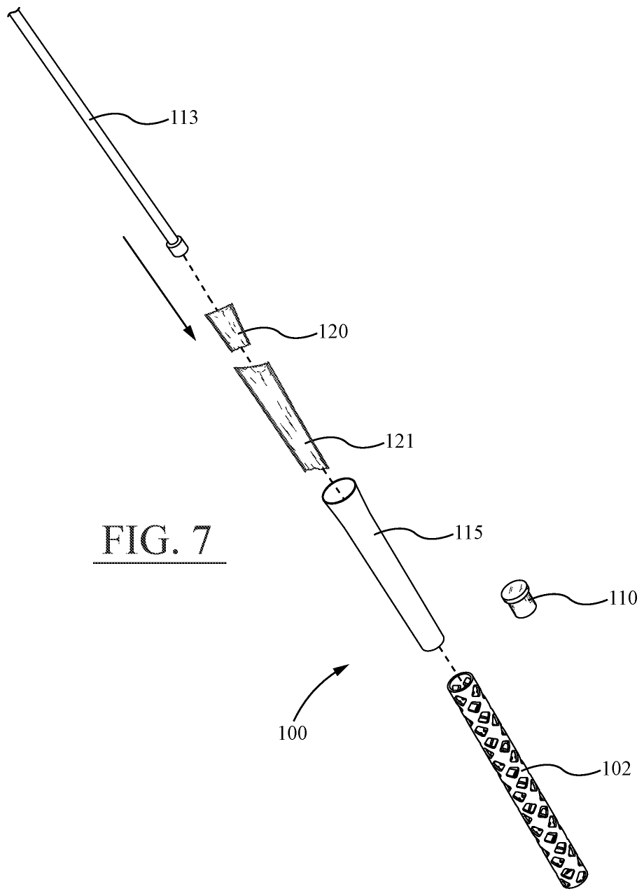

FIG. 7 is an exploded view depicting the process of loading a tube housing a packet for generating a sterilizing agent, according to some embodiments. The process depicted in FIG. 7 can be adapted for mass production. A metal shield insert 115 may be inserted into an empty tube 102. Then packets 120 and 121 may be placed in the larger funnel end of insert 115 and a ramrod 113 is used to pack the packets 120 and 121 inside the tube 102 up against a sealed end of the tube 102. In some embodiments, packet 121 is packed adjacent to the sealed and packet 120 is packed behind packet 121. After insertion of the ramrod 113, the insert 115 is pulled back over the ramrod 113, leaving behind packets 120 and 121. Then the cap 110 may be applied to secure the packets 120 and 121.

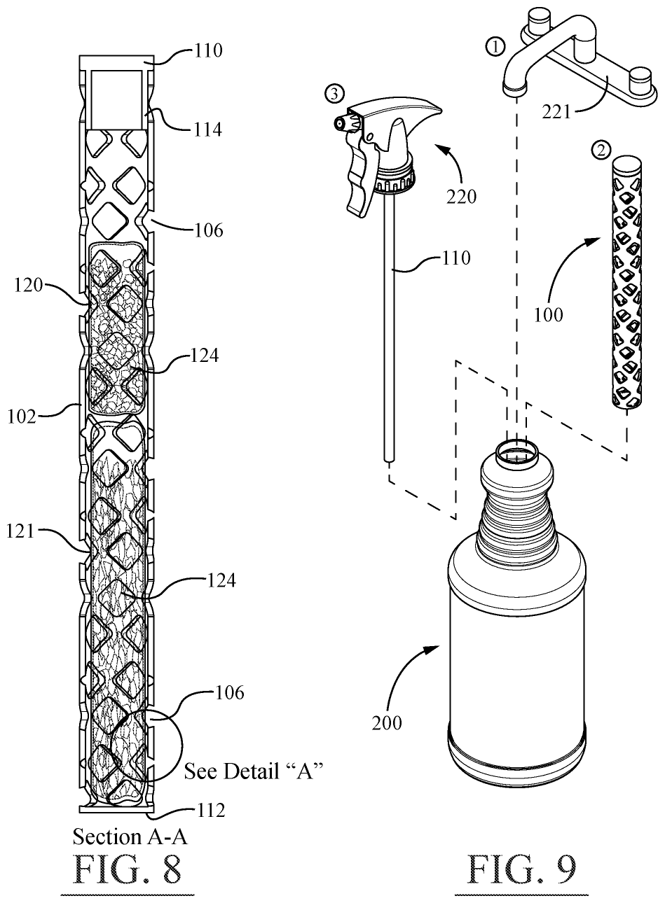

FIG. 3 is a side view depicting a cross section cut of a tube housing a packet for generating a sterilizing agent depicted in FIG. 8, which is an internal view of a packet situated within a tube, according to some embodiments. As illustrated, reactant packet 121 may be proximate to the holes 106 in wall 104. Holes 106 allow for passage of an initiating agent, for example water, into the tube 102 and packet 121 to cause the generation of a sterilizing agent. The resulting sterilizing agent is released into the surrounding water and passes back out of the holes 106.

FIG. 9 graphically depicts use of a tube housing a packet for generating a sterilizing agent, according to some embodiments. Tap water 221 may be added to a spray bottle 200. In some embodiments, the bottle 200 is reusable. In some embodiments, the water 221 is added up to a fill line indicated on the bottle 200.

After the bottle 200 is filled with water 221, the apparatus 100 is inserted into the bottle 200. Alternatively, apparatus 100 may be pre-loaded into the bottle 200 before the addition of water 221. Trigger top 220 is immediately screwed onto the bottle 200 after introduction of water 221, trapping the apparatus 100 in the bottle 200 while the reactant begins a reaction with water 221.

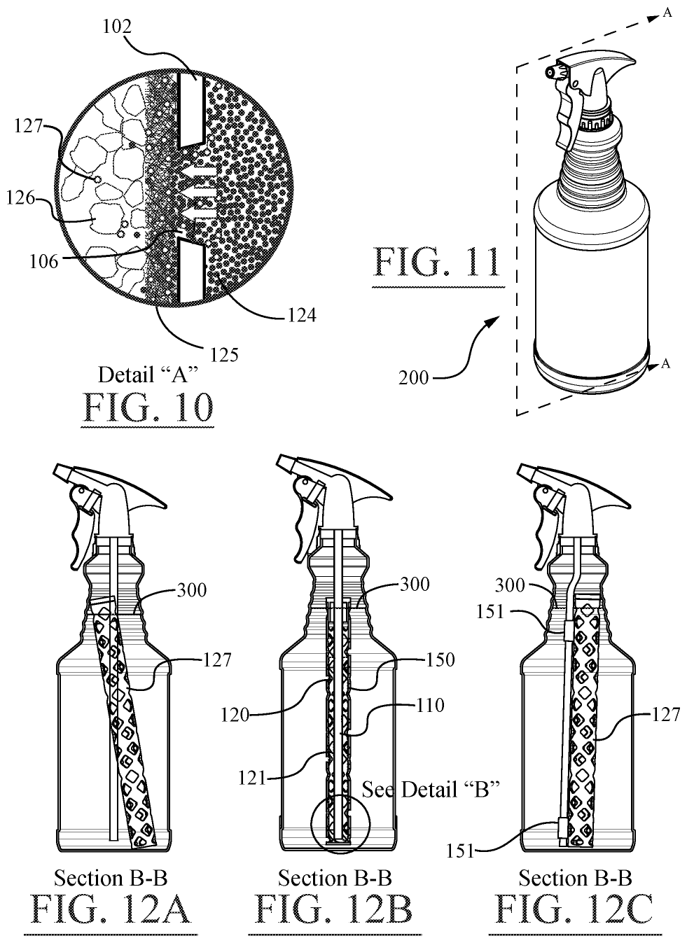

FIG. 10A is a mock-up microscopic view of an initiating agent entering a pouch containing a reactant, according to some embodiments. As illustrated, the packet wall 125 is composed of a sufficiently porous material to allow the passage of the initiating agent 126, for example water. The initiating agent also passes through holes 106 in the tube 102. The initiating agent 126 then interacts with the reactant 124 to generate a sterilizing agent 127.

FIG. 10B is a mock-up microscopic view of a generated sterilizing agent exiting a pouch containing a reactant, according to some embodiments. As illustrated, the tube 102 is configured to allow the passage of the generated sterilizing agent while retaining the reactant 124. The sterilizing agent mixes with the remaining water to form an aqueous cleaning solution. The cleaning solution may be drawn by a straw of a trigger top of a spray bottle and applied to a surface needing disinfection, cleaning, sanitizing, and the like.

FIG. 11 depicts the section cut line of a spray bottle 200 for the views depicted in FIGS. 12A-12C. FIG. 12A depicts a first embodiment of a tube housing a packet for generating a sterilizing agent situated in a conventional spray bottle. The apparatus 100 is depicted inside a filled spray bottle with the trigger sprayer screwed on. This is the state of a deployed apparatus that's either in the process of generating the sterilizing agent, finished generating the sterilizing agent and ready for use, or (minus the liquid) is what the apparatus 100 looks like once used up. The apparatus 100 is substantially submerged under the water line 300. There is enough room within the bottle for the apparatus 100 to be inside the bottle but still allow the straw from the trigger sprayer to pass by adjacent to the apparatus 100 to gain access to the bottom of the bottle for a full drain of liquid product within the bottle.

FIG. 12B depicts a second embodiment of a tube housing a packet for generating a sterilizing agent situated in a spray bottle. In the second embodiment there is an interior shaft pathway 150 down the center of the apparatus so that the entire apparatus can be preloaded on the trigger sprayer straw 111. The pathway 150 may run through packets 120 and 121. Alternatively, pathway 150 may be formed from a solid material and packets 120 and 121 may be inserted around the pathway 150.

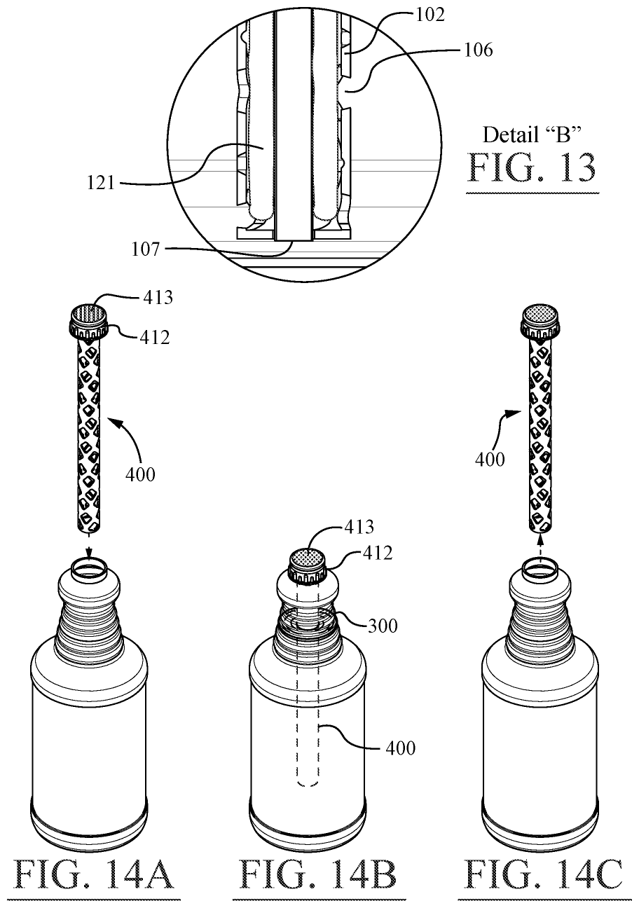

FIG. 13 is a close-up side view of the portion the embodiment of FIG. 12B labeled “Detail B.” In this embodiment, the straw protrudes out the cartridge bottom 107 so that the straw can pull in liquid from the bottom of the bottle. In this embodiment, the packet 121 may be inserted by placing the trigger on the filled bottle if the apparatus is attached to a straw of the trigger. Once removed from an empty bottle the apparatus may then be simply pulled off the straw and recycled. Another new apparatus is then slipped onto the straw for the next batch for sterilizing agent to be produced.

FIG. 12C depicts a third embodiment of a tube housing a packet for generating a sterilizing agent situated in a spray bottle. At least one clip 151 may be employed to secure the apparatus 100 to the straw. The clip 151 may be an integrated component of the straw. In other embodiments, the clip 151 is a separate component that attaches to the straw and the apparatus 100.

FIGS. 14A, 14B, and 14C depict a pH indicator cap as part of a tube housing a pouch for generating a sterilizing agent, according to some embodiments. When the bottle is filled with water and the apparatus 400 is screwed down, a color change may occur on the PH indicator 413 situated on cap 412. For example, the color may change from red to green. The color change gives the user a visual indication that the dwell time required for a full reaction between the reactant and initiating agent has happened and the product is ready to use. In some embodiments, the cap 412 may be removed and recycled with the apparatus 400 so that a conventional trigger sprayer top can be applied to the bottle.

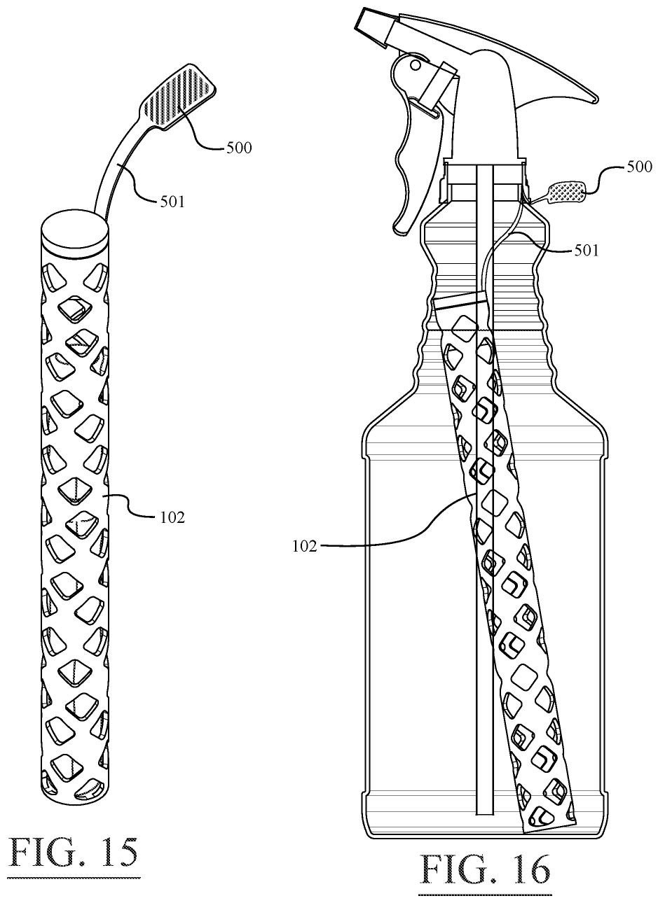

FIG. 15 depicts a pH indicator tethered to a tube housing a packet for generating a sterilizing agent and FIG. 16 depicts a pH indicator tethered to a tube housing a packet for generating a sterilizing agent situated within a spray bottle, according to some embodiments. A tether 501 may be attached to the tube 102. The tether 501 includes a pH indicator 500 at end opposite of the tube 102. The tether 501 may be made of an abosrbent material such that the liquid within the spray bottle would wick up to the indicator 500. When a level of sterilizing agent producing a desired pH is reached inside the bottle the pH indicator 500 is configured to change color to indicate the sterilizing agent is ready for use. The pH indicator 500 may be composed of sufficiently thin material so that the trigger top of the bottle can be screwed down successfully and the wicking material of the tether 501 is still is able get past the threads of the bottle. Once the bottle is empty and the apparatus is ready to remove, a user can grab the tether 501 and pull the apparatus out.

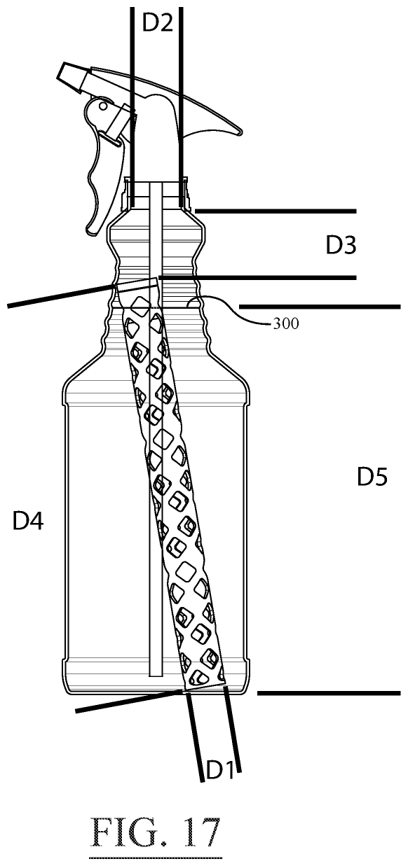

FIG. 17 depicts labeled dimensions of a conventional spray bottle relative to an exemplary embodiment of the disclosed apparatus. The dimensions indicate comparative distances. In some embodiments, the width of the apparatus D1 is a smaller distance compared to the width of the spray bottle neck D2 to allow for insertion and removal of the apparatus. Distance D3 must be sufficiently long to accommodate the trigger mechanism above the apparatus. Apparatus length D4 must be less than or equal to distance D5, the distance from fill line 300 to the bottom of the spray bottle, so that, regardless of the end of the apparatus inserted into the bottle, the reactant packet is submerged in the initiating agent, for example water. The apparatus length D4 must also be long enough to insure that the apparatus does not fall within the bottle and become stuck.

The disclosed apparatus may be used to generate sterilizing agents for a variety of uses. In some embodiments, chlorine dioxide is generated for use as a sterilizing agent. The generated chlorine dioxide mixes with the surrounding water to for a sprayable cleaning solution. The structure of the apparatus allows for easy removal from a spray bottle and subsequent recycling.

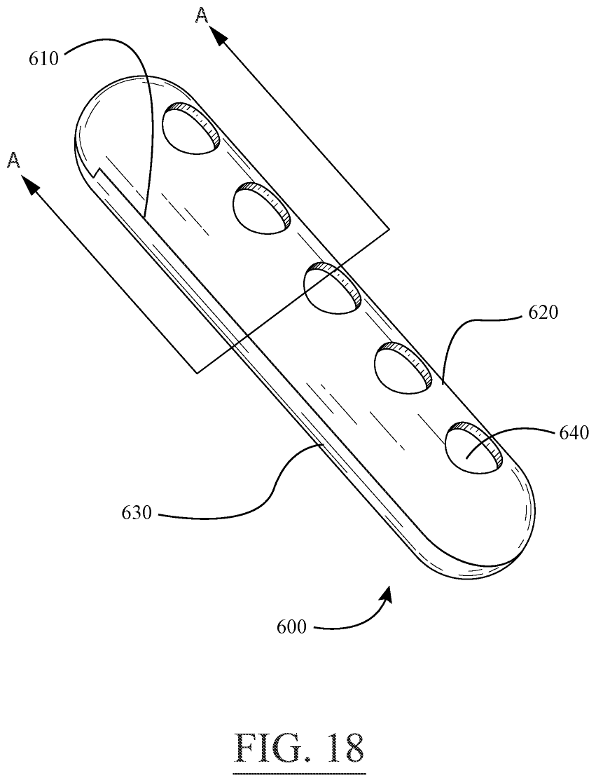

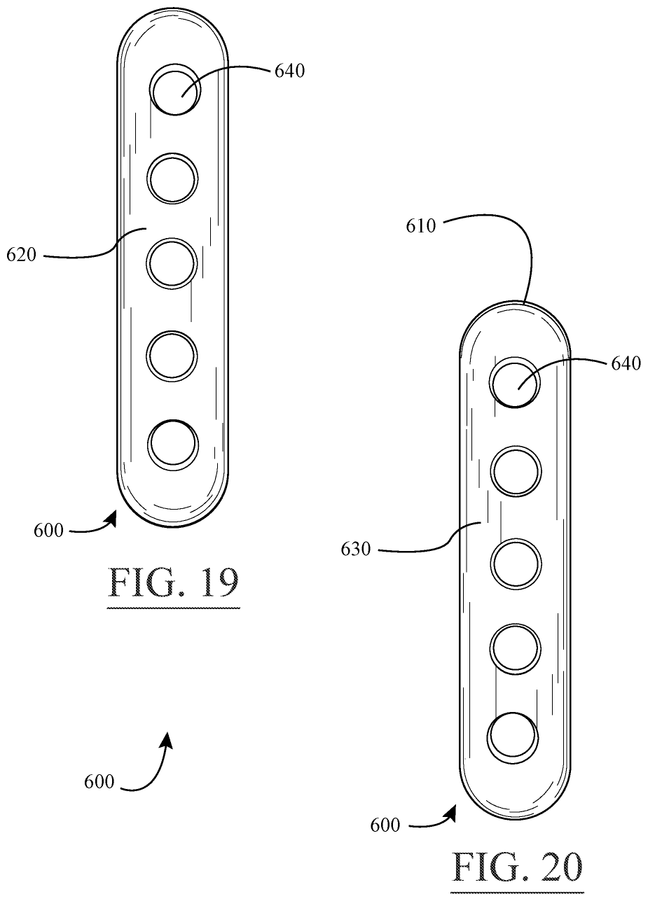

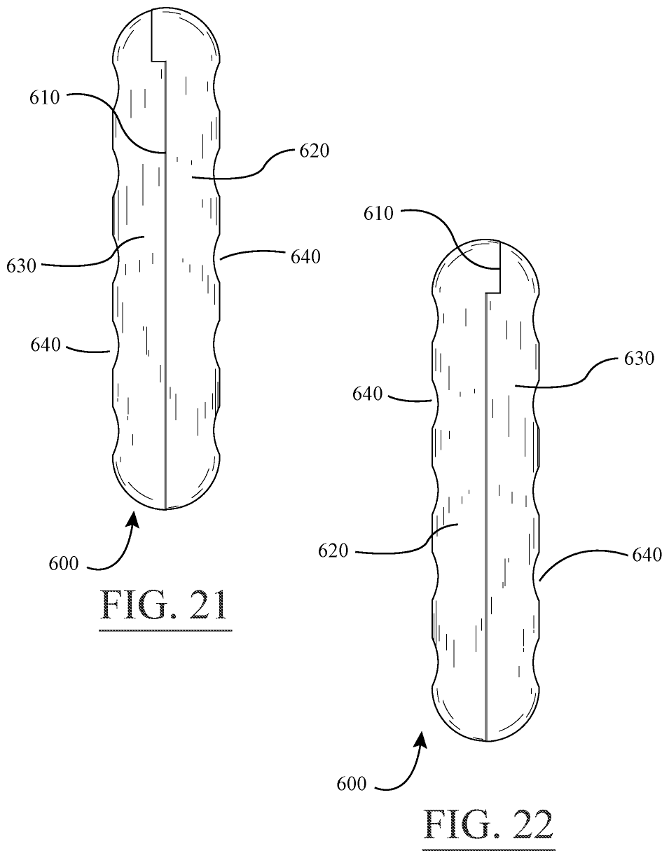

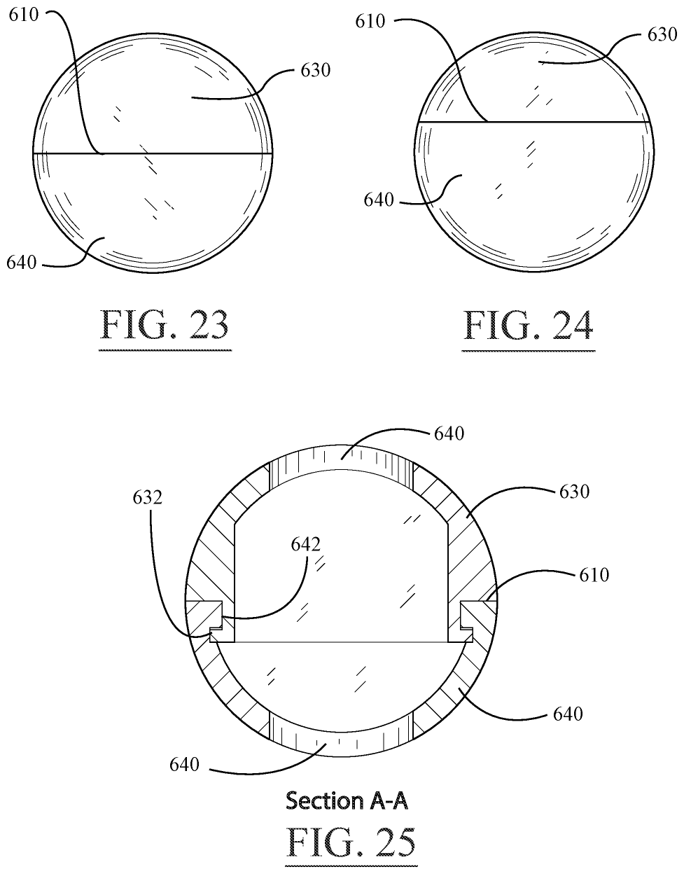

FIGS. 18-25 depict a capsule 600 for housing a packet 121 for generating a sterilizing agent, according to some embodiments. In some embodiments, the capsule 600 includes two components joined together along a seam 610, for example top component 620 and bottom component 630. The two components may be separated to allow insertion of a packet 121. In some embodiments, the capsule 600 is composed of a plastic material. In some embodiments, the capsule 600 is sized and shaped to fit within the neck of a spray bottle, or other similarly ported vessel.

Components 620 and 630 include at least one hole 640. Each hole 640 is configured to allow liquid or humid air to enter the capsule 600. In some embodiments, the holes 640 of component 620 and 630 are aligned when the components are secured together. In some embodiments, holes 640 are situated along a longitudinal axis of the capsule 600.

The capsule 600 (containing a packet 121) may be inserted into a spray bottle, either before or after the bottle is filled with water. Water may then enter the capsule 600 via the holes 640. The water will then react with the reactant in the packet 121. The resulting product will exist the capsule 600 via the holes 640 and mix with the surrounding water.

Alternatively, the capsule 600 (containing a packet 121) may be placed on a dry surface. Humid air may then enter the capsule 600 via the holes 640. In such embodiments, the packet 121 may be composed of an outer material that is permeable to humid air. The humid air will then react with the reactant in the packet 121. The resulting product will exist the capsule 600 via the holes 640 and mix with the surrounding environment.

FIG. 25 is a section view of the capsule 600 of FIG. 18. The section view depicts the slide rails 632 used to secure the capsule 600 in a locked position. The slide rails 632 are discussed in further detail below with regards to FIGS. 27 and 28. Aligned holes 640 can also be seen at the top and bottom of the section view.

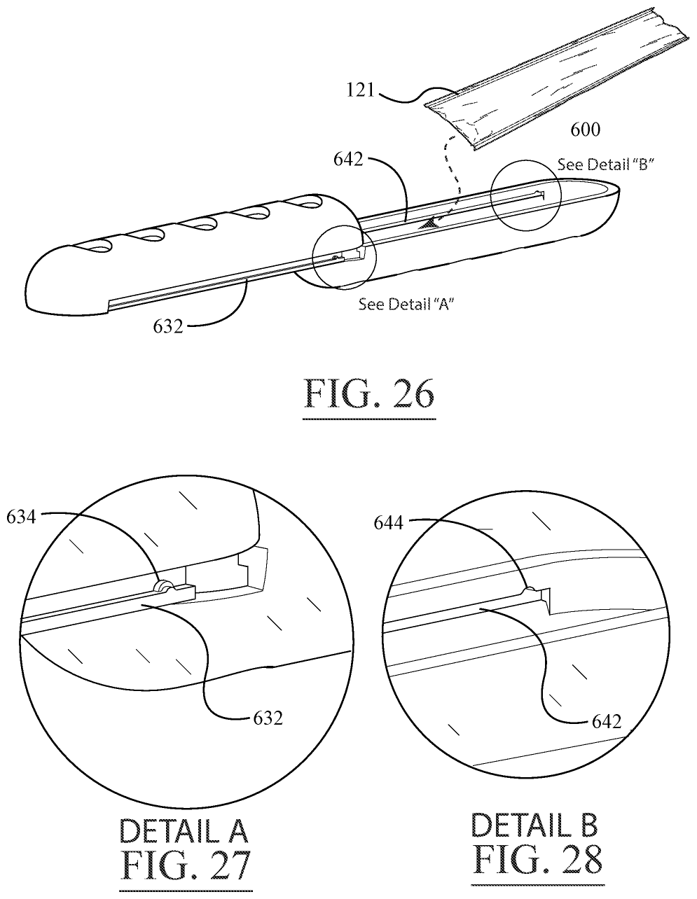

FIG. 26 is a perspective view of a packet 121 being loaded into an open capsule 600, according to some embodiments. In some embodiments, the capsule 600 is sized to allow for expansion of the packet 121 within the capsule 600. In some embodiments, the capsule 600 is cylindrically shaped. Packet 121 may include a reactant as described above.

When closed, the holes 640 of the capsule 600 allows for the entry of liquid or humid air to activate the reactant within the packet 121. Once activated, the packet 121 may swell. In some embodiments, the capsule 600 is sized such that the packet 121 will swell enough that the swollen packet 121 provides sufficient pressure against the inside of the closed capsule 600 to reinforce the interlocked slide rails 632, further securing the capsule 600 in a closed position.

FIG. 27 is an enlarged view of a detent 634 of a rail 632 for locking the capsule 600, according to some embodiments. FIG. 28 is an enlarged view of a notch 644 of a channel 642 for locking the capsule 600, according to some embodiments.

Top component 620 may include two slide rails 632. Each rail 632 may be situated on opposing edges of component 620. Each rail 632 fits into a corresponding channel 642 on the lower component 630 of the capsule 600. The detent 634 is configured to provide pressure and friction as a rail 632 slides into a channel 642. The lower component 620 with channel 642 may be composed of a plastic material that provides sufficient flexibility to allow detent 634 to push along the channel 642 until the detent 634 hits the notch 644.

Once the top component 620 slides through the channel 642 the detent 634 on each rail 632 will pop into the corresponding notch 644 of each channel 642, semi-permanently securing the two components together with a friction fit. In other words, the connection of the detent 634 and notch 644 is not a permanent snap feature. Upon application of a sufficient force, the components may be separated to open the capsule 600 and the packet 121 may be removed and/or replaced.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

What is claimed is:

1. An apparatus comprising:
a hollow cylinder having a closed end and an open end, the cylinder comprising a plurality of holes disposed on a body of the cylinder; and
at least one packet disposed within the cylinder, the at least one packet containing a reactant.

2. The apparatus of claim 1, further comprising a packet containing a desiccant, the packet disposed within the cylinder.

3. The apparatus of claim 1, wherein the plurality of holes allows entry of a liquid into the cylinder.

4. The apparatus of claim 3, wherein the cylinder is sufficiently rigid to prevent expansion the packet beyond the body of the cylinder after entry of the liquid.

5. The apparatus of claim 1, wherein the cylinder is configured to be insertable through a neck of a spray bottle.

6. The apparatus of claim 5, wherein the cylinder is mounted to a straw of the spray bottle.

7. The apparatus of claim 6, wherein the cylinder is mounted to the straw with at least one clip.

8. The apparatus of claim 1, further comprising a pH indicator attached to the cylinder.

9. The apparatus of claim 8, wherein the pH indicator is a cap removably attached to the open end of the cylinder.

10. The apparatus of claim 8, wherein the pH indicator is a tether attached to a cap removably attached to the open end of the cylinder.

11. The apparatus of claim 1, further comprising a cap removably attached to the open end of the cylinder.

12. A method of preparing a disinfecting agent comprising:
selecting a hollow cylinder having a closed end and an open end, the cylinder comprising a plurality of holes disposed on a body of the cylinder;
inserting and securing a packet within the cylinder, the packet containing a reactant; and
introducing a liquid into the cylinder.

13. The method of claim 12, further comprising inserting the cylinder into a spray bottle.

14. The method of claim 13, wherein the cylinder is inserted through a neck of the spray bottle.

15. The method of claim 13, further comprising securing the cylinder within the spray bottle.

16. The method of claim 15, wherein the cylinder is secured with at least one clip.

17. The method of claim 15, wherein the cylinder is secured to a straw of the spray bottle.

18. The method of claim 17, wherein the disinfecting agent is released through the straw out of the spray bottle.

19. The method of claim 12, wherein the liquid is introduced through at least some of the plurality of holes.

20. The method of claim 12, wherein the cylinder is sufficiently rigid to prevent expansion the packet beyond the body of the cylinder after introducing of the liquid.

21. The method of claim 12, wherein the liquid is water.
